(19) United States
(12) Patent Application Publication
(10) Pub. No.: US 2026/0216117 A1
(43) Pub. Date: Jul. 30, 2026

(54) COMPOSITION AND METHOD FOR CLEANING AND DEBRIDING SKIN WOUNDS AND/OR ULCERS

(71) Applicant: UNIVERSIDAD DEL DESARROLLO, Santiago (CL)

(72) Inventors: María Belén OLIVARES, Santiago (CL); Christina SCHUH, Santiago (CL); Paola Rossana CAMPODÓNICO GALDAMES, Santiago (CL)

(21) Appl. No.: 19/149,198

(22) PCT Filed: Jan. 18, 2023
(86) PCT No.: PCT/CL2023/050005

§ 371 Date: Jul. 18, 2025

(51) Int. Cl. A61K 31/205; A61K 31/17; A61P 17/02

(52) U.S. Cl. CPC A61K 31/205; A61K 31/17; A61P 17/02

(57) ABSTRACT

The present invention relates to a medical composition and device containing said composition which comprises a eutectic mixture that includes betaine, urea, and water in an appropriate ratio for use in the cleansing and debridement of wounds and/or skin ulcers.

### TECHNICAL FIELD

The present invention falls within the technical field of the pharmaceutical industry. Specifically, it relates to compositions and methods useful for cleaning and debriding wounds or ulcers of human or animal skin, and particularly for the cleaning and debridement of chronic and/or infected wounds. The present invention refers to a composition comprising a mixture of betaine, urea, and water in specific proportions and ranges.

### BACKGROUND INFORMATION

Wounds are defined as a disruption of the normal structure and function of the skin. Acute wounds that occur in a normal physiological context progress through the various stages of healing until complete resolution. In contrast, chronic wounds are those that occur with pathophysiological conditions that prevent progress towards closure or resolution. Skin ulcers are chronic wounds that, after a period of around four weeks, have not healed and represent a cause of morbidity and mortality that has significantly increased in recent years.

The healing process of chronic wounds is slow and demanding. Furthermore, there is a marked imbalance between the demand and availability of clinical personnel that affects the effectiveness of addressing this important public health issue. Additionally, 50% of patients with chronic wounds require at least one hospitalization. The treatment and resolution of wounds and skin ulcers is becoming increasingly challenging due to risk factors that complicate it, such as the aging population and associated comorbidities (obesity and diabetes, among others), as well as local risk factors of the skin in post-surgical, burned, and/or bedridden patients, which are mainly associated with the lack of irrigation of the damaged area. Cutaneous ulcers are characterized by a prolonged inflammatory state that translates into an elevated state of phagocytosis and apoptosis phenomena by neutrophils and macrophages at the injured site. As a result of this intense inflammatory cellular activity, the production of eschar occurs, which is a yellowish, viscous tissue composed of fibrin, and other protein materials along with micro debris. It has been shown that the removal of this devitalized material contributes to the prevention of wound infection, a process known as debridement, which is performed by various agents in the standard treatment of wounds.

The inability of the skin tissue to achieve wound closure and restore the protective capacity of the skin leads to critical colonization of the tissues by microorganisms that, within a few hours, can establish themselves in the form of biofilm (biofilms) on the viable and non-viable matrix (eschar or necrotic plaques) of the affected tissues. The presence of microbial biofilms becomes a factor that prevents closure and worsens the healing prognosis of the wound, leading to or worsening the state of infection, being one of the local factors that most impact the closure process, in addition to compromising the underlying tissues and promoting eventual septicemia. The combination of tissue-biofilm-eschar-necrotic represents a complex polymeric matrix and a significant clinical challenge. Therefore, this is the primary barrier to the healing of chronic wounds, and failure to eliminate it in the chronic wound healing process poses a high risk of progression to a state of generalized infection.

The common approach to managing wounds and skin ulcers focuses on two aspects, depending on the clinical assessment by the treating physician or nurse: i) application of local cleaning strategies and ii) promotion of the skin restoration, to achieve complete closure or healing of the wound. In the general protocols applied to lesions with eschar and/or necrotic plaque, these two aspects translate into applying what is known as advanced or moist healing, and their cleaning procedures are based on the debridement of the lesion with procedures and/or products that generate the least possible impact on the healing processes and, ultimately, protection of the internal environment of the lesion. Additionally, there are also adjunctive therapies that contribute to promoting cell proliferation and wound closure.

In systematic reviews of the reported clinical evidence, the lack of therapeutic efficacy of the use of antimicrobial active ingredients is recognized, in comparison with the mere application of dressing systems that act physically and non-pharmacologically in treatment. This finding is related to the fact that bacterial colonization in the structures of the lesion in chronic wounds is primarily in the form of resistant biofilms adhered to devitalized tissues. The therapeutic failure of cleaning protocols involving products including biocidal antiseptics, traditional antimicrobials, and even broad-spectrum antibiotics, is explained because such strategies target the microorganism physically, without recognizing that it is embedded in a physically and chemically protected environment that prevents the arrival of these products.

Based on the above, a series of products have been developed with the aim of altering the physical properties of the microbial biofilm associated with devitalized eschar in wounds. Among these are membrane disruptors such as biguanides and surfactants, such as chlorhexidine, polyhexamethylene biguanide commonly known as polyhexanide or PHMB, alone or in combination with undecyl-enamidopropyl betaine (Prontosan™, currently the first choice in cleaning chronic wounds due to its biocompatibility), up to more complex surfactants such as peptide-based ones with antibacterial activity and other biomolecules contained in natural bio-capsules such as exosomes. On the other hand, and less recommended due to their toxicity, are quaternary ammonium compounds such as cetrimide. Another target for attacking the biofilm is to affect its water-retaining capacity. In this regard, generating osmotic stress causes dehydration of the matrix, which prevents the structuring of water channels, essential for bacterial communication and viability. In this area, products applied with the aim of debriding tissue by generating osmotic stress could be acting on this front. In this line are medical honeys (Manuka Honey™, UlmoPlus™, or those described in EP2670413, CN100522258, EP2077864, WO2007/045931, EP2040669) which, due to their high sugar content, represent hypertonic mixtures, a topical strategy currently indicated for wound treatment. However, the main disadvantage of medical honeys is that they are complex mixtures of sugars, pollen, proteins, etc., which make them variable and unpredictable in composition and, therefore, difficult to characterize, standardize, and control, both in production and in post-marketing surveillance phases. Furthermore, medical honeys contain protein elements with high immunogenic potential, both derived from bee secretions and of plant origin.

Debridement of tissue is a very relevant procedure within the cleaning strategy as it not only removes devitalized tissue that promotes an inflammatory state but also contributes to the physical removal and attack of the biofilm. This reduces the bacterial load present in the wound, especially in lesions with abundant sloughy or necrotic tissue, as these are heavily colonized with aerobic and anaerobic bacteria. Unlike surgical debridement, which is performed in the operating room for the most critical cases presenting infection in deeper tissues such as bone or cartilage, medical debridement is commonly performed during dressings in primary care and/or home care settings. Common protocols for this type of debridement consider i) mechanical action (by scraping with tools and/or with gauze) or ii) enzymatic debridement, by applying external enzymes, or by occluding the area so that the wound's own enzymes act, for example, as described in patent documents EP1907022 and EP1965763. Enzymatic products are generally not recommended for being cost-ineffective and, moreover, require a strict cold chain to maintain stability. For its part, autolytic debridement is not recommended in cases of critically colonized wounds because it requires a long occlusion time, increasing the risk of infection. Another form of debridement is achieved by occluding the area with a hyperosmotic product (hydrogels, honeys) that, through osmotic action, attracts fluid from lymphatic and blood vessels to the surface of the wound, promoting the detachment of sloughy or necrotic tissues; however, these are inefficient procedures and present high variability.

From the above, it follows that both the debridement process and cleaning with currently available products fail in eliminating microbial remnants and preventing the recolonization of wounds with a biofilm that hinders their proper healing.

### SUMMARY OF THE INVENTION

The present invention relates to a composition for cleaning and debriding wounds and/or skin ulcers, comprising a eutectic mixture composed of betaine, urea, and water in a molar ratio of between 1:1.5:1 and 1:4.5:2.5. This new composition cleans the wound by inducing the debridement of devitalized tissue and helps eradicate microbial biofilms in infected wounds.

The composition of the present invention is better than existing formulations because it acts in a dual manner as a disruptor of microbial biofilms and as a debridement agent for devitalized skin tissues; preferably, it does not contain conventional biocides that may generate antimicrobial resistance; its eutectic liquid state promotes greater penetration than other solid (dressings) and semi-solid (gels) debridement agents; it has a simple composition and formulation, and therefore easy production and quality control; and it is stable at ambient or storage temperatures (between 10-40° C.).

Preferably, the eutectic mixture of the present invention is composed of betaine, urea, and water in a molar ratio of 1:1.5:2. In a preferred embodiment, the composition is in liquid or semi-solid form.

The present invention also relates to a medical device for cleaning and debriding wounds and/or skin ulcers, comprising a eutectic mixture composed of betaine, urea, and water in a molar ratio of between 1:1.5:1 to 1:4.5:2.5, preferably 1:1.5:2, and an appropriate support, where said appropriate support is selected from the group consisting of a dressing, patch, bandage, gauze, fabric, tulle, compress, film, gel, hydrogel, foam, cream, ointment, hydrocolloid, synthetic or natural polymer, and adhesive tape.

The present invention also relates to a eutectic mixture composed of betaine, urea, and water in a molar ratio of between 1:1.5:1 to 1:4.5:2.5, preferably 1:1.5:2, for use in the cleaning and debridement of wounds and/or skin ulcers.

The present invention also relates to the use of a composition comprising a eutectic mixture composed of betaine, urea, and water in a molar ratio of between 1:1.5:1 to 1:4.5:2.5, preferably 1:1.5:2, for the manufacture of a medicine or a medical device useful for the cleaning and debridement of wounds and/or skin ulcers.

The present invention also relates to a method for cleaning and debriding wounds and/or skin ulcers that comprises obtaining a composition that includes a eutectic mixture composed of betaine, urea, and water in a molar ratio between 1:1.5:1 to 1:4.5:2.5, preferably 1:1.5:2, and applying said composition to a wound and/or skin ulcer.

In a preferred embodiment of the method of the present invention, the composition is applied to the wound or skin ulcer in a liquid, semisolid form, or as part of a medical device.

### BRIEF DESCRIPTION OF THE DRAWINGS

FIG. 1 shows a graph with the in vitro debridement activity of different compositions: enzymatic and various NADES. The debridement capacity was measured as a percentage of the structural breakdown of a collagen-fibrin matrix based on total absorbance over time for a period of 3 hours. FIG. 1A shows a comparison of the structural breakdown kinetics of the protein fibrils absorbance at 350 nm measured every 15 minutes for 3 hours. FIG. 1B shows a comparison of the percentage of matrix disorganization at the end of 3 hours of treatment.

FIG. 2 shows the debridement activity in vitro of different compositions of the NADES formulated from various proportions of betaine, urea and water. The debonding capacity was measured as a percentage of the disintegration of a collagen-fibrin matrix based on total absorbance over time for a period of 3 hours. FIG. 2A shows a comparison of the kinetics of disassembly of the protein fibrils absorbance at 350 nm measured every 15 minutes over 3 hours. FIG. 2B shows a comparison of the percentage of matrix disorganization at the end of 3 hours of treatment.

FIG. 3 shows the results of the evaluation of the disruptive capacity of bacterial biofilms by BU002 in vitro. The disruptive capacity of bacterial biofilms was determined through the measurement of the antibacterial activity of BU002 in biofilms of Pseudomonas aeruginosa and Staphylococcus aureus grown on collagen matrices, determining the proportion of viable bacteria by flow cytometry.

FIG. 4 shows the results of the evaluation of the disruptive capacity of bacterial biofilms by BU002 in vivo. The ability to eliminate bacterial biofilms in wounds was determined by performing a bacterial count in wound biopsies by the colony-forming unit per gram of tissue CFU/g counting method obtained from wounds in mice.

### DETAILED DESCRIPTION OF THE INVENTION

This invention relates to a composition that simultaneously allows for the debridement of devitalized and necrotic tissue and the eradication of microbial biofilms present in wounds and/or ulcers of human or animal skin. This composition comprises a eutectic mixture composed of betaine, urea, and water, in a specific molar ratio range.

A eutectic mixture, more specifically, a natural deep eutectic solution is known in the scientific literature as NADES for its acronym in English (Natural Deep Eutectic Solvent). These products are natural because they are formed by a mixture of molecules of natural origin, and they are recognized as new chemical entities, therefore, with new applications (Sanchez, B., et al. Gutmann's Donor and Acceptor Numbers for Ionic Liquids and Deep Eutectic Solvents. Front Chem 2022; 10:861379). NADES are formed by the mixture of solid substances that, in certain proportions, have the ability to interact intermolecularly forming a supramolecular network, achieved through hydrogen bond-type interactions and generating a liquid eutectic mixture, since its melting point is much lower than that of the original components. NADES resemble in many ways the monomers of a polymer, but are joined by non-covalent bonds, which is advantageous as a biomaterial because, unlike polymers, the non-covalent bonding of NADES gives their small and biocompatible molecular blocks the potential to be easily metabolized and/or biotransformed.

Currently, NADES are being widely researched in biomedical applications since, due to the origin of their components, they project a very favorable profile of biocompatibility and safety. For example, it is known that compositions of NADES can be useful as solvents to prevent the degradation of β-lactam antibiotics (Olivares, B., et al. A Natural Deep 15 Eutectic Solvent Formulated to Stabilize β-Lactam Antibiotics. Sci Rep 2018; 8:14900) or to enhance their activity (Olivares, B., et al. Betaine-urea deep eutectic solvent improves imipenem antibiotic activity. J Mol Liq 2022; 350:118551). However, different types of NADES and different formulations of the same NADES result in compositions with different types of physicochemical properties that make them interact with other molecules in a specific way, effects that cannot be easily predicted without experimentation. Additionally, different types of NADES and formulations of the same NADES result in different stabilities at room temperature and viscosities that determine the rheological characteristics that provide fluidity and specific diffusion to certain formulations.

Surprisingly, the composition of the present invention comprising a eutectic mixture of betaine, urea, and water has proven to be highly effective for the cleaning and debridement of wounds and/or skin ulcers.

The composition of the present invention is, in a preferred embodiment, a hyperosmolar liquid composed of betaine, urea, and water. The range of molar proportions of each of those components in the formula is selected with the aim of achieving maximum stability of the composition at room temperature and a viscosity that allows the rheological characteristics to provide fluidity and diffusion to the composition.

This composition has shown to have antibiofilm activity against Pseudomonas aeruginosa, Staphylococcus aureus, and Staphylococcus epidermidis, which are the prevalent microorganisms in skin wounds (see examples later). These results demonstrate that the present composition is a liquid with high penetration capability, able to reach and penetrate the matrix of the biofilm formed by bacterial cells that settle very intricate among the fibers of the connective tissue. Once in contact with the biofilm, the present composition generates osmotic stress in situ, similar to that attributed to medical honeys, but with greater penetration capacity than these.

In this composition, each urea molecule is found strongly bound to a betaine molecule, as demonstrated by nuclear magnetic resonance studies and molecular dynamics studies. In this way, and as occurs in other NADES, the physicochemical properties of its components are modified. It was evidenced that the high nucleophilicity of urea is radically modified, becoming harmless even to molecules that are highly sensitive to its attack. In this sense, betaine acts in the present composition as a modulator of the chaotropic and denaturing power of the urea molecule. Betaine is a natural hydrophilic molecule, widely used in pharmaceutical and cosmetic products, including those approved for wound washing (polyhexanide-betaine or PHMB). But the betaine found in the composition of this invention, unlike the mentioned products, is not linked to a hydrophobic alkyl chain that gives rise to a surfactant, with limitations of use due to toxicity in cell membranes, but rather the composition of the present invention turns out to be a purely polar compound, even with a greater dipole moment than water. With this, the present composition is, without the need for other excipients, very water-soluble, a fundamental quality for both its therapeutic target and its bio-elimination. Additionally, the hydrophilic nature is an advantage in this application, given that the subepidermal tissues are mostly hydrophilic, just like the polymeric matrix of the biofilm it is intended to intercept.

On the other hand, the hyperosmolarity characteristics of the composition of the present invention are not only advantageous in the action against the biofilm for its antibacterial action, but also in the debridement action of non-viable tissues. Unlike honeys or hydrogels, in the present composition there is the proteolytic potential of urea, which could act on the abundant eschar and/or necrotic plaque, stimulating the wound bed as collagenases do in enzymatic debridement, but unlike the products enzymatic, the present composition is very stable at room temperature and has a much lower production cost.

All technical and scientific terms used to describe the present invention have the same meaning understood by a person with basic knowledge in the relevant technical field. However, to define with greater clarity the scope of the invention, below is a list of the terminology used in this description and its meaning.

The term “wound” should be understood as those open wounds where the skin of a subject is torn, cut, pierced, or any other superficial injury or condition on the skin of that subject, where the skin may include injuries in the epidermis, dermis, hypodermis, or subcutaneous fat tissue, or it may even refer to deeper open wounds that reach the muscle layer. Such wounds include acute, subacute, chronic, dehiscent, traumatic wounds, lacerations, surgical wounds, abrasions, contusions, burns, diabetic ulcers, pressure ulcers, among others, and may or may not be infected. The term “subject” can refer to humans or animals.

“Wound cleaning” should be understood as the hygienization of a wound that includes the removal of bacterial biofilms, foreign bodies, and physical contaminants, but does not include the disinfection (inhibition or death) of microorganisms in the affected area. That is to say, it should be understood that the eutectic mixture of the present invention allows for the disruption of bacterial biofilms and therefore the cleaning of the wound, but it does not exert an inhibitory or lethal effect directly on the microorganisms, as an antibiotic does, for example. However, this does not prevent the composition of the present invention from additionally comprising other active compounds that exert an antibacterial effect such as antibiotics, biocides, or disinfectants, although it is preferable that it does not contain them to avoid the generation of antimicrobial resistance.

The term “medication” refers to any substance or combination of substances that is presented as having properties for the treatment or prevention of diseases in a subject or that can be used in a subject or administered to a subject in order to restore, correct, or modify physiological functions by exerting a pharmacological, immunological, or metabolic action, or to establish a medical diagnosis. An “active ingredient” or “active substance” is any substance or mixture of substances intended for the manufacture of a medicine and which, when used in its production, becomes an active component of that medicine intended to exert a pharmacological, immunological, or metabolic action in order to restore, correct, or modify physiological functions, or to establish a diagnosis.

The term “medical device” or “health product” for the purposes of the present invention refers to any instrument, implant, reagent, material, or other article intended for use in a subject, which allows for the prevention, treatment, or relief of wounds, and which does not exert its main action inside or on the surface of the subject by pharmacological, immunological, or metabolic mechanisms, but whose function may contribute to such mechanisms. For example, the present invention considers a medical device that includes the composition comprising a eutectic mixture composed of betaine, urea, and water, where the composition exerts the pharmacological effect.

A first object of the present invention comprises a composition that allows for the simultaneous cleaning and debridement of skin wounds, which includes a eutectic mixture composed of betaine (also known as trimethylglycine, TMG, or by its IUPAC name 2-(trimethylazaniumyl)acetate), urea (also known as carbamide or by its IUPAC name diaminomethanone), and water in a molar ratio between 1:1.5:1 to 1:4.5:2.5, preferably a molar ratio of 1:1.5:2.

The composition of the present invention is applied superficially or topically to a wound in a subject. Said composition may be in liquid, solid, semisolid form, or a mixture thereof, including aqueous or oily suspensions, among others. Preferably, the composition is in liquid form to facilitate the cleaning and debridement of wounds, or in a semisolid form such as, for example, a gel. “Liquid” should be understood as a fluid composition with a wide range of viscosities and densities. Preferably, the composition has a viscosity between 85-115 cP (mPa·s) at 25° C.

The composition may additionally include a pharmaceutically acceptable excipient. Such an excipient should be understood as any component of the pharmaceutical composition other than the active compound, and may refer to a diluent, stabilizer, binder, disintegrant, gelling agent, thickener, among others, or a mixture of them. These excipients are known in the state of the art, such as those reported, for example, in Allen L, Popovich N, Ansel H. (2011). Ansel's Pharmaceutical Dosage Forms and Drug Delivery Systems. 9th edition. Lippincott Williams & Wilkins, and Rowe R, Sheskey P, Quinn M. (2009). Handbook of Pharmaceutical Excipients. 6th edition. Pharmaceutical Press, included here by reference, and not limited to those mentioned there. The agents and specific compounds for each of these functions are evident to a person with knowledge of the state of the art and can be found in multiple publications such as those mentioned above.

A second object of the present invention relates to a medical device for cleaning and debridement of wounds, comprising a eutectic mixture composed of betaine, urea, and water in a molar ratio between 1:1.5:1 and 1:4.5:2.5, preferably in a molar ratio of 1:1.5:2.

The medical device of the present invention may have different shapes and sizes. The medical device additionally comprises a suitable support for the composition, where such supports may be patches, dressings, bandages, gauzes, fabrics, tulle, pads, films, gels, hydrogels, foams, creams, ointments, hydrocolloids, synthetic or natural polymers, adhesive tapes, among others. The eutectic mixture of the present invention may be embedded, or absorbed in the medical device, or the medical device may be impregnated, wet, moistened, or soaked with the eutectic mixture. For example, it may be a gauze that contains the eutectic mixture composed of betaine, urea, and water embedded in it.

The medical device contains a sufficient amount of the eutectic mixture so that it can be applied over the entire wound bed, adequately covering all damaged and/or infected tissue. This medical device can be applied as many times as required by the affected subject until the desired results are achieved.

A third object of the present invention relates to a eutectic mixture of betaine, urea, and water in a molar ratio between 1:1.5:1 and 1:4.5:2.5 for use in the cleaning and debridement of wounds. Preferably, the molar ratio of betaine, urea, and water is 1:1.5:2. The eutectic mixture is used by applying it to a wound, in a sufficient amount and as many times as necessary to achieve the cleaning and debridement of the wound.

A fourth object of the present invention relates to the use of a composition comprising a eutectic mixture composed of betaine, urea, and water in a molar ratio between 1:1.5:1 to 1:4.5:2.5 for the manufacture of a drug or a medical device useful for cleaning and debridement of wounds and/or skin ulcers. Preferably, the molar ratio between betaine, urea, and water is 1:1.5:2.

A fifth object of the present invention is a method for the cleaning and debridement of wounds that comprises obtaining a composition that includes a eutectic mixture composed of betaine, urea, and water in a molar ratio between 1:1.5:1 to 1:4.5:2.5, preferably 1:1.5:2, and apply a sufficient amount of said composition to a wound and/or skin ulcer to achieve the desired effect. Such a sufficient amount should be understood as an adequate quantity to cover the wound bed, and it can be applied to the wound as many times as necessary to obtain the desired effect.

In a preferred embodiment of the use or method, the composition is applied in liquid form or as part of a medical device, such as those described in the present invention.

### EXAMPLES

The following examples are intended to illustrate the invention and its preferred embodiments, but under no circumstances should they be considered to limit the scope of the invention, which will be defined by the wording of the claims that are attached hereto.

It should be noted that, throughout the text, the acronyms BU00X (e.g. BU002, BU003, etc.) correspond to different embodiments of the composition of the present invention.

### Example 1. Evaluation of the Debridement Effect of Different NADES in a Model of Deconstruction of Protein Fibril Matrices In Vitro

Different types of NADES were evaluated for their ability to disrupt biofilms in a model of artificial matrices made of collagen and fibrin, which has proven to be a predictive model of the effect of different compounds on the stability of the protein structure (Shi, L., et al., Study on the debridement efficacy of formulated enzymatic wound debriding agents by in vitro assessment using artificial wound eschar and by an in vivo pig model. Wound Repair Regen 2009; 17(6):853-862; Stuart, K., et al, Influence of chondroitin sulfate on collagen gel structure and mechanical properties at physiologically relevant levels. Biopolymers 2008; 89:841-851). The methodology for forming the protein matrix corresponds to an adaptation of the protocols in the cited articles as described below: 200 μl collagen I (rat tail Gibco®) was mixed with 60 μl PBS 10× and 45 μL Fibrinogen (Merck) 25 mg/ml. Then, 5 μL of NaOH 1 N and 7.6 μl of thrombin (Sigma) in a 10 U/ml solution in a solution of calcium chloride 40 mM were added. The mixture was aliquoted in volumes of 50 μl in a 96-well plate. It was incubated at 37° C. for one hour and then for 24 hours at 4° C.

The absorbance of the matrix was measured at 350 nm, as it directly relates to the disorganization or denaturation of the matrix after applying the various treatments under evaluation. The absorbance data were normalized to the total content in the matrix to obtain the percentage of disorganization of the matrix.

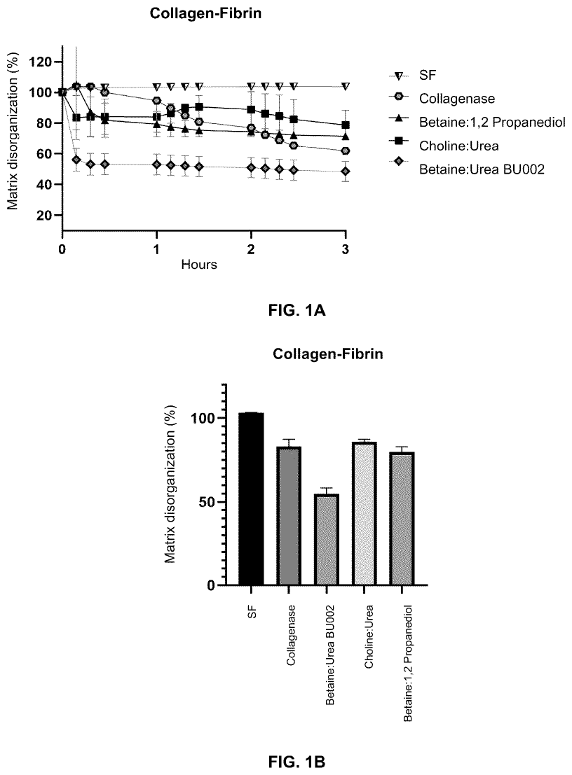

FIG. 1 shows the results of the evaluation of the kinetics and the extent of the debridement effect of different NADES in a model of disorganization of protein fibril matrices in vitro. In FIG. 1A and FIG. 1B, the comparative effect of betaine: urea (1:1.5:2 betaine, urea, water BU002) with the NADES Choline: Urea (1:1.5:1.5 choline chloride: urea: water) and the NADES Betaine: 1,2 Propanediol: (1:1.5:0.3 betaine: 1,2 propanediol: water) is shown. In the same graphs, the effect of physiological saline (negative control), collagenase (positive control) is observed. Specifically, in FIG. 1A, the comparison of the kinetics of disorganization of artificial matrices of fibers induced by different NADES measured every hour for 3 hours after applying 50 μl of physiological saline (SF), Collagenase 500 μg/ml, NADES betaine:urea:water (BU002), NADES choline:urea, and NADES betaine: 1,2 Propanediol is shown. In FIG. 1B, the comparison of the percentage of disorganization of artificial matrices of protein fibers at the end of 3 hours of treatment after applying 50 μl of physiological saline (SF), Collagenase 500 μg/ml, NADES BU002, NADES choline:urea, and NADES betaine: 1,2 Propanediol is shown.

The results of the comparison of these different NADES formed from the combination of different molecules that have been reported as forming this type of solvent indicate that the formulation from betaine:urea:water is superior in its penetration and disorganization capacity of collagen and fibrin fibrils much faster than that demonstrated by enzymes such as collagenase (FIG. 1A). Furthermore, the debridement effect of NADES formulated from betaine:urea:water is greater when compared to other NADES containing betaine or containing urea in their composition (FIGS. 1A and 1B).

### Example 2: Evaluation of the Debridement Effect of Different Formulations of the NADES Formed from the Mixture of Betaine, Urea, and Water

As noted earlier, different formulations of the same NADES can result in compositions with different types of physicochemical properties with different effects and interactions with biological matrices and in different stabilities.

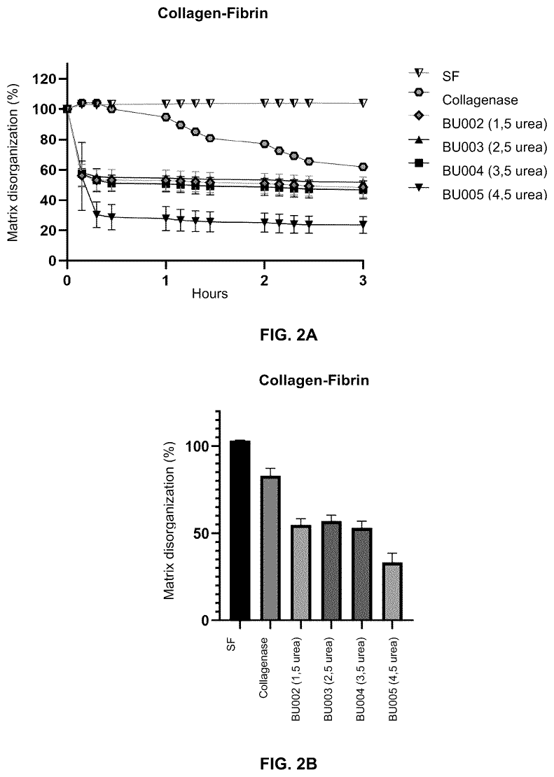

For this reason, the ability of different formulations of betaine, urea, and water obtained by the heating method at 70° C. and magnetic stirring in a round-bottom flask at 200 rpm for 60 min was evaluated in the penetration and disorganization of collagen and elastin fibrils. The results of this evaluation are shown in FIGS. 2A and 2B. FIG. 2A shows the results of the comparison of the kinetics of disorganization of artificial matrices of protein fibers induced by different formulations of betaine, urea, and water, measured every hour for 3 hours after applying 50 μl of physiological saline SF (sodium chloride 0.9%), Collagenase 500 μg/ml, NADES betaine:urea:water 1:1.5:2 (BU002 (1.5 urea)), NADES betaine:urea:water 1:2.5:2 (BU003 (2.5 urea)), NADES betaine:urea:water (1:3.5:2) (BU004 (3.5 urea)), and NADES betaine:urea:water 1:4.5:2) (BU005 (4.5 urea)). In FIG. 2B, the comparison of the percentage of disorganization of artificial matrices of protein fibers induced by different formulations of betaine, urea, and water at the end of 3 hours of treatment after applying 50 μl of physiological saline (SF) (sodium chloride 0.9%), Collagenase 500 μg/ml, NADES betaine:urea:water 1:1.5:2 (BU002 (1.5 urea)), NADES betaine:urea:water 1:2.5:2 (BU003 (2.5 urea)), NADES betaine:urea:water 1:3.5:2 (BU004 (3.5 urea)), and NADES betaine:urea:water 1:4.5:2 (BU005 (4.5 urea)) is shown.

The results demonstrate that the range of molar proportions of urea that encompass 1.5 (BU002); 2.5 (BU003) and 3.5 (BU004) have similar effects in the in vitro debridement assay. Although the molar ratio of urea 4.5 (BU005) is higher, the following assays continued with the composition containing a molar ratio of 2 (BU002) due to its greater long-term stability. The evaluated proportions correspond to those of maximum stability of the composition at room temperature and a viscosity that allows the rheological characteristics that provide fluidity and diffusion for topical treatment in wounds.

### Example 3. Evaluation of the Disruptive Capacity of Bacterial Biofilms by BU002 In Vitro

The composition encompassed in this invention is a hydrophilic, hyperosmotic fluid composed of a supramolecular network of betaine and urea that as such, can penetrate between the fibers of the tissue to affect the wound microenvironment by two mechanisms: i) the dehydration of the biofilm matrix, affecting its water-retaining capacity with its consequent disorganization and ii) by the proteolytic nature of urea, which affects the protein component of the biofilm, interfering with the processes of adhesion, communication, and pathogenesis of virulent proteins. Furthermore, the same hyperosmolar and proteolytic character of urea controlled by betaine confers a deep debriding action and therefore could act more effectively than current osmotic-type products (hydrogels or medicinal honeys) in the process of debridement of devitalized tissue.

The disruptive capacity of bacterial biofilms was determined through the measurement of the antibacterial activity of composition BU002 in biofilms of Pseudomonas aeruginosa (ATCC-27853) and Staphylococcus aureus (ATCC 29213) grown on collagen matrices (Alvarez, S., et al. Bacterial adhesion to collagens: implications for biofilm formation and disease progression in the oral cavity. Crit Rev Microbiol 2022; 48:1: 83-95), using the following protocol adapted from Price, B. L. et al. Development of a Novel Collagen Wound Model To Simulate the Activity and Distribution of Antimicrobials in Soft Tissue during Diabetic Foot Infection. Antimicrob. Agents Chemother 2016; 60 (11): 25 μl of an inoculum of 106 bacteria obtained from a logarithmic phase culture were seeded in 50 μl collagen gels. These were obtained according to the following protocol: 200 μl of collagen I (rat tail Gibco®) was mixed with 300 μl of sterile distilled water and 60 μL of 10×PBS while keeping the mixture cold on ice. Finally, 5 μl of 1N NaOH was added and quickly transferred 50 μl into 96-well plates. After incubating at 37° C. for one hour, the gels were kept at 4° C. for 24 hours before being inoculated. After obtaining 48-hour mature biofilms, treatments were applied: physiological saline (0.9% sodium chloride) and BU002. These were maintained for 24 hours and the bacterial count was performed by flow cytometry using live/dead staining with SytoBC/Propidium iodide, determining the proportion of viable bacteria. The results indicate that BU002 has an antibiofilm activity against Pseudomonas aeruginosa and Staphylococcus aureus which are the prevalent microorganisms in skin wounds (FIG. 3). These results indicate that BU002 is a liquid with high penetration capacity of reaching and penetrating the biofilm matrix formed by bacterial cells that are intricately lodged between the fibers of connective tissue. Once in contact with the biofilm, the composition of the present invention generates osmotic stress in situ, similar to that attributed to medical honeys, but with greater penetration capacity than these.

### Example 4. Evaluation of Antibiofilm Efficacy in Human Ex Vivo Model

The in vivo models of bacterial biofilm formation in wounds are a model closer to the structure and framework of connective fibers present in clinical ulcer models. A murine model of type I diabetes induced by Streptozotocin (200 mg/Kg) in C57BL/6 mice aged 10 weeks and weighing 20-25 grams of both sexes was employed, as described by Ezquer, F., et al. Endovenous Administration of Bone Marrow-Derived Multipotent Mesenchymal Stromal Cells Prevents Renal Failure in Diabetic Mice. Biol Blood Marrow Transplant 2009; 15(11):1354-65 and Ezquer, M., et al. Intravitreal administration of multipotent mesenchymal stromal cells triggers a cytoprotective microenvironment in the retina of diabetic mice. Stem Cell Res Ther 2016; 16; 7:42, with certain modifications. The same murine model has been reported to exhibit the characteristics of hyperglycemia that hinder healing and generate a deficient immune response, both factors that promote the establishment of bacterial biofilms in wounds (Chen, C. Y., et al. Exosomal DMBT1 from human urine-derived stem cells facilitates diabetic wound repair by promoting angiogenesis. Theranostics 2018; 8(6): 1607-1623; Krishnan, N., et al. Effect of biogenic silver nanocubes on matrix metalloproteinases 2 and 9 expressions in hyperglycemic skin injury and its impact in early wound healing in streptozotocin-induced diabetic mice. Mater Sci Eng C Mater Biol Appl 2018; 1; 91: 146-152). After 15 days of hyperglycemia, the mice were shaved on their backs, and a full-thick skin wound was induced using a biopsy puncturing instrument (6 mm). To avoid wound contraction, a silicone splint (Grace Bio-Labs) was used, (external diameter 14 mm, internal 7 mm) with cyanoacrylate-based glue and 4 sutures (Vicryl™ 5-0). 24 hours after the induction of the wound, bacteria were inoculated into them by applying 30 μl of a solution of 1×10\hat{ }8 CFU/ml of P. aeruginosa (ATCC-27853) and S. aureus (ATCC 29213) and the wound was covered with Tegaderm™. After 48 hours post inoculation, treatments were applied every 48 hours for 7 days (4 times).

The ability to eradicate the biofilm was evaluated by measuring the average difference in CFU/g between groups: treated with saline or with BU002 in wound biopsies that were suspended in 300 μl of PBS and agitated at 950 rpm for 40 min at 15° C. Then, 100 μl of the suspension was diluted and plated in triplicate for colony counting on plates according to the conventional method, in Chromoagar® medium.

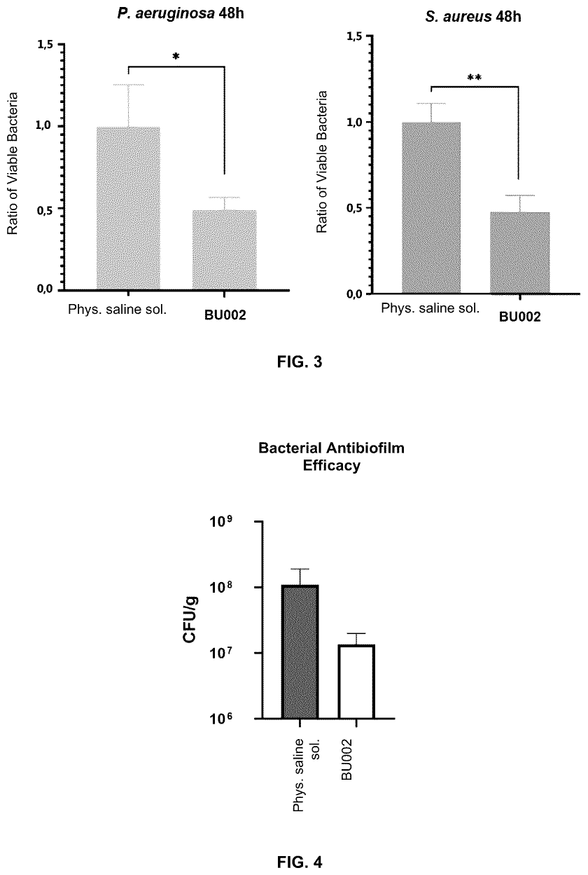

In FIG. 4, the bacterial count in wound biopsies is shown using the colony-forming unit count per gram of tissue method. It is observed that BU002 has an antibiofilm effect, reducing by approximately 10 times the viability of adherent bacteria that form 15 a 48-hour maturity biofilm.

1. A composition for cleaning and debridement of wounds and/or skin ulcers, the composition comprising a eutectic mixture comprising betaine, urea, and water.

2. The composition according to claim 1, wherein the eutectic mixture is in a molar ratio of betaine:urea:water between 1:1.5:1 and 1:4.5:3.5.

3. The composition according to claim 1, wherein it is in liquid or semi-solid form.

4. A composition for cleaning and debridement of wounds and/or skin ulcers, the composition comprising a eutectic mixture comprising betaine, urea, and an excipient.

5. The composition of claim 4, wherein the excipient is a diluent.

6. The composition of claim 5, wherein the eutectic mixture and the diluent are in a molar ratio between 1:1.5:1 and 1:4.5:3.5.

7. A medical device for cleaning and debridement of wounds and/or skin ulcers, comprising the composition of claim 1 and an appropriate support.

8. The medical device of claim 7, wherein the appropriate support is selected from the group consisting of a dressing, patch, bandage, gauze, cloth, tulle, compress, film, gel, hydrogel, foam, cream, ointment, hydrocolloid, synthetic or natural polymer, and adhesive tape.

9. The medical device of claim 8, wherein the appropriate support is embedded, soaked, moistened, wetted, or impregnated with the eutectic mixture.

10. A method for using the composition of claim 1 for cleaning and debridement of wounds and/or skin ulcers.

11. A method of using the composition of claim 1 for the manufacture of a medicament or medical device useful for cleaning and debridement of wounds and/or skin ulcers.

12. A method for cleaning and debridement of wounds and/or skin ulcers, comprising applying the composition of claim 1 to a wound and/or skin ulcer.

13. The method of claim 12, wherein the composition is applied in liquid or semi-solid form, or as part of a medical device.

14. A medical device for cleaning and debridement of wounds and/or skin ulcers, comprising the composition of claim 6 and an appropriate support.

15. The medical device of claim 14, wherein the appropriate support is selected from the group consisting of a dressing, patch, bandage, gauze, cloth, tulle, compress, film, gel, hydrogel, foam, cream, ointment, hydrocolloid, synthetic or natural polymer, and adhesive tape.

16. The medical device of claim 15, wherein the appropriate support is embedded, soaked, moistened, wetted, or impregnated with the eutectic mixture.

17. A method for using the composition of claim 6 for cleaning and debridement of wounds and/or skin ulcers.

18. A method of using the composition of claim 6 for the manufacture of a medicament or medical device useful for cleaning and debridement of wounds and/or skin ulcers.

19. A method for cleaning and debridement of wounds and/or skin ulcers, comprising applying the composition of claim 6 to a wound and/or skin ulcer.

20. The method of claim 19, wherein the composition is applied in liquid or semi-solid form, or as part of a medical device.
